(19) United States
(12) Patent Application Publication
(10) Pub. No.: US 2013/0143278 A1
(43) Pub. Date: Jun. 6, 2013

(54) RECYCLE OF LEACHATE DURING LIGNOCELLULOSIC CONVERSION PROCESSES

(71) Applicants: Jeffrey S. Tolan, Ottawa (CA); Daphne Wahnon, Ottawa (CA); David Geros, Ottawa (CA)

(72) Inventors: Jeffrey S. Tolan, Ottawa (CA); Daphne Wahnon, Ottawa (CA); David Geros, Ottawa (CA)

(73) Assignee: Iogen Energy Corporation, Ottawa, ON (CA)

(21) Appl. No.: 13/816,267

(22) PCT Filed: Aug. 11, 2011
(86) PCT No.: PCT/CA2011/050491

§ 371 Date: Feb. 11, 2013

Related U.S. Application Data
(60) Provisional application No. 61/372,493, filed on Aug. 11, 2010.

(51) Int. Cl. C12P 7/14; C12P 19/14

(52) U.S. Cl. CPC C12P 7/14; C12P 19/14
USPC 435/99; 435/162; 435/158; 435/139; 435/144; 435/137; 435/145; 435/136; 435/146; 435/140

(57) ABSTRACT

The present invention provides a process for producing fermentable sugar or a fermentation product from a lignocellulosic feedstock. The process comprises leaching the lignocellulosic feedstock with an aqueous solution to remove at least potassium salts from the lignocellulosic feedstock and without significantly hydrolyzing hemicellulose and cellulose, thereby producing a leached feedstock and leachate. The leachate is removed from the leachate and concentrated. The leached feedstock is hydrolyzed to produce fermentable sugar, which may be fermented to produce a fermentation broth comprising the fermentation product. The concentrated leachate is recirculated to one or more stages in the process involving alkali addition to adjust the pH of a process stream.

### TECHNICAL FIELD

The present invention relates to an improved process for producing fermentable sugar from a lignocellulosic feedstock.

### BACKGROUND

Plant cell walls consist mainly of the large biopolymers cellulose, hemicellulose, lignin and pectin. Cellulose consists of D-glucose units linked together in linear chains via beta-1,4 glycosidic bonds. Hemicellulose consists primarily of a linear xylan backbone comprising D-xylose units linked together via beta-1,4 glycosidic bonds and numerous side chains linked to the xylose units via beta-1,2 or beta-1,3 glycosidic or ester bonds (e.g. L-arabinose, acetic acid, ferulic acid, etc.).

Lignocellulosic feedstock is a term commonly used to describe plant-derived biomass comprising cellulose, hemicellulose and lignin. Much attention and effort has been applied in recent years to the production of fuels and chemicals, primarily ethanol, from lignocellulosic feedstocks, such as agricultural wastes and forestry wastes, due to their low cost and wide availability. These agricultural and forestry wastes are typically burned and landfilled; thus, using these lignocellulosic feedstocks for ethanol production offers an attractive alternative to disposal. Yet another advantage of these feedstocks is that the lignin byproduct, which remains after the cellulose conversion process, can be used as a fuel to power the process instead of fossil fuels. Several studies have concluded that, when the entire production and consumption cycle is taken into account, the use of ethanol produced from cellulose generates close to zero greenhouse gases.

In comparison, fuel ethanol from feedstocks such as corn starch, sugar cane and sugar beets suffers from the limitation that these feedstocks are already in use as a food source for animals and humans. A further disadvantage of the use of these feedstocks is that fossil fuels are used in the conversion processes. Thus, these processes have only a limited impact on reducing greenhouse gases.

Lignocellulosic feedstocks have also been considered for producing other products besides ethanol. For example, lactic acid has received much attention in recent years for the production of biodegradable lactide polymers. It is expected that this biodegradable polymer, produced from renewable resources, will partially replace various petrochemical-based polymers in applications ranging from packaging to clothing (van Maris et al., 2004, Microbial Export of Lactic and 3-Hydroxypropanoic Acid: Implications for Industrial Fermentation Processes, In Metabolic engineering of pyruvate metabolism in Saccharomyces cerevisiae, Ed. Van Maris, ppg 79-97).

The first chemical processing step for converting lignocellulosic feedstock to ethanol or other fermentation products involves hydrolysis of the cellulose and hemicellulose polymers to sugar monomers, such as glucose and xylose, which can be converted to ethanol or other fermentation products in a subsequent fermentation step. Hydrolysis of the cellulose and hemicellulose can be achieved with a single-step chemical treatment or with a two-step process with milder chemical pretreatment followed by enzymatic hydrolysis of the pretreated lignocellulosic feedstock with cellulase enzymes.

In the single-step chemical treatment, the lignocellulosic feedstock is contacted with a strong acid or alkali under conditions sufficient to hydrolyze both the cellulose and hemicellulose components of the feedstock to sugar monomers.

In the two-step chemi-enzymatic hydrolysis process, the lignocellulosic feedstock is first subjected to a pretreatment under conditions that are similar to, but milder than, those in the single-step acid or alkali hydrolysis process. The purpose of the pretreatment is to increase the cellulose surface area and convert the fibrous feedstock to a muddy texture, with limited conversion of the cellulose to glucose. If the pretreatment is conducted with acid, the hemicellulose component of the feedstock is hydrolyzed to xylose, arabinose, galactose and mannose. The resulting hydrolyzate, which is enriched in pentose sugars derived from the hemicellulose, may be separated from the solids and used in a subsequent fermentation process to convert the pentose sugars to ethanol or other products.

After the pretreatment step, the cellulose is subjected to enzymatic hydrolysis with one or more cellulase enzymes such as exo-cellobiohydrolases (CBH), endoglucanases (EG) and beta-glucosidases. The CBH and EG enzymes catalyze the hydrolysis of the cellulose (β-1,4-D-glucan linkages). The CBH enzymes, CBHI and CBHII, act on the ends of the glucose polymers in cellulose microfibrils and liberate cellobiose, while the EG enzymes act at random locations on the cellulose. Together, the cellulase enzymes hydrolyze cellulose to cellobiose, which, in turn, is hydrolyzed to glucose by beta-glucosidase (beta-G).

If glucose is the predominant sugar present in the hydrolyzate, the fermentation is typically carried out with a Saccharomyces spp. strain. However, if the hydrolyzate comprises significant proportions of xylose and arabinose carried through from the pretreatment, the fermentation is conducted with a microbe that naturally contains, or has been engineered to contain, the ability to ferment xylose and/or arabinose to ethanol or other product(s). Examples of microbes that have been genetically modified to ferment xylose include recombinant Saccharomyces strains into which has been inserted either (a) the xylose reductase (XR) and xylitol dehydrogenase (XDH) genes from Pichia stipitis (U.S. Pat. Nos. 5,789,210, 5,866,382, 6,582,944 and 7,527,927 and EP 450 530) or (b) fungal or bacterial xylose isomerase (XI) gene (U.S. Pat. Nos. 6,475,768 and 7,622,284).

Each stage for producing fermentable sugar from the feedstock is typically carried out at a pH range at which the chemical or biological reaction operates most efficiently. The pH of the incoming feedstock is between about 6.0 and 8.0 and then is decreased with acid to a pH between about 0.5 and 2.0, which is a conventional pH range for acid pretreatment (see WO 2006/128304). After acid pretreatment, alkali is added to the acidic, pretreated feedstock to achieve the optimal pH range of 4.5 to 5.5 for cellulase enzymes. The pH of the glucose stream resulting from enzymatic hydrolysis may be subsequently adjusted to a value that is amenable to most fermentations and this is usually between 4 and 5.5 for the yeast that are commonly used in this stage, such as Saccharomyces cerevisiae.

One drawback of conventional processes is that significant amounts of acid and alkali are required to attain the pH ranges that are considered optimal for each stage. The high chemical demand for carrying out the pH adjustments at various stages of the process can significantly increase the cost. Compounding this, the addition of acid or alkali during the pH adjustments produces inorganic salts as a consequence of the neutralization of alkali or acid added in previous stages. This further increases the cost of the process as these salts must be processed and disposed of.

Acid pretreatment is one stage of the process that has a particularly high acid demand. The feedstock has a pH of between 6 and 8 due to the presence of the alkali minerals such as potassium carbonate, sodium carbonate, calcium carbonate and magnesium carbonate, and thus requires the addition of significant amounts of acid to adjust the pH of the feedstock down to values between 0.5 and 2.0. The minerals have a neutralizing effect on the pretreatment acid (Esteghlalian et al., 1997, Bioresource Technology, 59:129-136). For instance, sulfuric acid reacts with the cations of the carbonate salts during pretreatment to form potassium sulfate, sodium sulfate, calcium sulfate and magnesium sulfate. Bisulfate salts form as the pH is lowered further. Due to the presence of these minerals, additional acid is required to overcome the resistance of the feedstock to changes in pH, which further contributes to the chemical requirements of this stage.

The most prevalent element in the feedstock that is a source of cations is potassium (See co-owned U.S. Pat. No. 7,585,652, which is incorporated herein by reference). Other elements in the feedstocks that are significant sources of cations include calcium, sodium, and magnesium, at concentrations of about 1/3, 1/7, and 1/10 that of potassium, respectively. Most of the potassium, calcium, sodium, and magnesium in the feedstocks is complexed with organic compounds, such as proteins or carboxylic acids, or exists in the form of oxides or oxlates. The feedstocks are slightly alkaline with this “excess” of cations, as the concentration of anions is low.

The pH adjustment conducted to increase the pH of the acidic, pretreated feedstock to between 4.5 and 5.5 with alkali prior to enzymatic hydrolysis with cellulase enzymes also contributes significantly to the high chemical demand of the process. This is compounded by the presence of acetic acid that arises from the hydrolysis of acetyl groups from hemicellulose during acid pretreatment. Notably, the pKa of acetic acid is 4.75 and, at a pH corresponding to its pKa, the buffering capacity of this weak acid is at its maximum. Thus, when the acidic, pretreated feedstock is increased from a pH between 0.5 and 2.0 to a pH between 4.5 and 5.5 for enzymatic hydrolysis, significant amounts of alkali must be added to overcome the buffering effect of this weak organic acid. High levels of alkali addition also produce large amounts of salts as the alkali reacts with the acid in the pretreated feedstock.

The pH adjustment prior to fermentation may also necessitate the addition of alkali to adjust the pH of the glucose stream to the optimal pH of the microbes. As acetate and acetic acid arising from acid pretreatment will also be present in the glucose stream, the buffering effect will again need to be overcome to adjust the pH.

WO 02/070753 (Griffin et al.) discloses a leaching process to remove alkali from lignocellulosic feedstocks, thereby decreasing the acid requirement for chemical treatment. The process includes contacting the feedstock with an aqueous solution of pH 3-9 to leach out the salts, protein, and other impurities. The leachate containing these soluble compounds is then removed from the feedstock. This process decreases the acid requirements in the subsequent pretreatment process, which can increase the yield of xylose after pretreatment.

U.S. Pat. No. 7,585,652 (Foody et al.) discloses leaching a lignocellulosic feedstock to remove potassium therefrom. The leachate may be concentrated by evaporation and/or reverse osmosis, clarified by microfiltration, plate and frame filtration or centrifugation and then separated from organics by ion exclusion with SMB to produce a fertilizer product.

U.S. Pat. No. 4,908,067 (Just) discloses a continuous counter-current leaching process with acid hydrolysis of wood chips. Just describes pre-soaking wood chips with acid or wetting them with water. If acid is utilized, any excess solution can be drained away for re-use. The presoaked feedstock is then continuously fed to a reactor loop of a tubular reactor where additional water is added to form a slurry. The slurry is then continuously subjected under pressure as it goes through several reactor loops in series. Each reactor loop is heated with a heat exchanger to raise the temperature of the pressurized slurry therein to a temperature sufficient for hydrolysis of the slurry to occur. The temperature of the slurry is maintained for sufficient time for leaching of the slurry to occur. After cooling and depressurization of the slurry, the solid portion of the slurry is separated from the slurry and passed to the next reactor loop of the tubular reactor. The liquor portion of the slurry containing sugar is recovered and a portion is injected to a preceding reactor loop of the tubular reactor.

Despite these efforts, there is a continuous need for more efficient and cost effective processes for producing fermentable sugar from lignocellulosic feedstock. In particular, there is a need in the art to further reduce acid and alkali demand during such processes.

### SUMMARY OF THE INVENTION

The present invention overcomes several disadvantages of the prior art by taking into account the difficulties encountered in steps carried out during the processing of lignocellulosic feedstock to obtain fermentable sugar.

It is an object of the invention to provide an improved method for producing fermentable sugar from a lignocellulosic feedstock.

According to a first aspect, the present invention provides a process for producing glucose from a lignocellulosic feedstock comprising the steps of:

(i) leaching the lignocellulosic feedstock with an aqueous solution to remove at least potassium salts from said lignocellulosic feedstock and without significantly hydrolyzing hemicellulose and cellulose, thereby producing a leached feedstock and leachate;
(ii) removing the leachate from leached feedstock, said leachate comprising at least potassium salt;
(iii) concentrating the leachate comprising the potassium salt to produce concentrated leachate;
(iv) pretreating the leached feedstock with acid to produce an acid pretreated lignocellulosic feedstock;
(v) adjusting the pH of the acid pretreated lignocellulosic feedstock by alkali addition to produce a pH adjusted feedstock having a pH between about 4 and about 6;
(vi) enzymatically hydrolyzing the pH adjusted feedstock with cellulase enzymes to produce a stream comprising at least glucose; and
(vii) recirculating the concentrated leachate comprising at least potassium salt to
the step of adjusting the pH of the acid pretreated lignocellulosic feedstock.

According to a second aspect of the invention, there is provided process for producing a fermentation product from a lignocellulosic feedstock comprising the steps of:

(i) leaching the lignocellulosic feedstock with an aqueous solution to remove at least potassium salts from said lignocellulosic feedstock and without significantly hydrolyzing hemicellulose and cellulose, thereby producing a leached feedstock and a leachate;
(ii) removing the leachate from leached feedstock, said leachate comprising at least potassium salt;
(iii) concentrating the leachate comprising the potassium salt to produce concentrated leachate;
(iv) pretreating the leached feedstock with acid to produce an acid pretreated lignocellulosic feedstock;
(v) optionally removing an aqueous stream from the acid pretreated lignocellulosic feedstock comprising the acid and at least xylose, increasing the pH of said aqueous stream to a pH between about 4.0 and 6.0 and then fermenting the xylose to produce a fermentation product;
(vi) adjusting the pH of the acid pretreated lignocellulosic feedstock by alkali addition to produce a pH adjusted feedstock having a pH between about 4 and about 6;
(vii) enzymatically hydrolyzing the neutralized feedstock with cellulase enzymes to produce a stream comprising at least glucose;
(viii) optionally increasing the pH of the stream comprising glucose to a pH between about 4.0 and about 6.0 prior to fermenting;
(ix) fermenting the glucose with microorganisms to produce the fermentation product; and
(x) recirculating the concentrated leachate comprising at least potassium salt to
(a) the step of adjusting the pH of the acid pretreated lignocellulosic feedstock;
(b) the step of increasing the pH of the stream comprising glucose;
(c) the aqueous stream comprising xylose prior to fermenting same; or
(d) a combination thereof.

According to a third aspect of the invention, there is provided a process for producing glucose from a lignocellulosic feedstock comprising the steps of:

(i) leaching the lignocellulosic feedstock with an aqueous solution to remove at least potassium salts from said lignocellulosic feedstock and without significantly hydrolyzing hemicellulose and cellulose, thereby producing a leached feedstock and leachate;
(ii) removing the leachate from leached feedstock, said leachate comprising at least potassium salt;
(iii) concentrating the leachate comprising the potassium salt to produce concentrated leachate;
(iv) pretreating the leached feedstock with acid to produce an acid pretreated lignocellulosic feedstock;
(v) adjusting the pH of the acid pretreated lignocellulosic feedstock by alkali addition to produce a pH adjusted feedstock having a pH between about 4 and about 6;
(vi) enzymatically hydrolyzing the pH adjusted feedstock with cellulase enzymes to produce a stream comprising at least glucose; and
(vii) recirculating the concentrated leachate comprising at least potassium salt to one or more stages of the process involving alkali addition to adjust the pH of a process stream.

The lignocellulosic feedstock may be selected from the group consisting of corn stover, soybean stover, corn cobs, rice straw, rice hulls, corn fiber, wheat straw, barley straw, canola straw, oat straw, sugar cane straw, oat hulls and combinations thereof. According to one embodiment of the invention, at least about 90% by weight of the feedstock particles in said lignocellulosic feedstock have a length less than between about 1/16 and about 6 in.

According to any of the foregoing aspects of the invention, the concentrated leachate may have a pH of between about 7.0 and about 11.0. Concentrating of the leachate may comprise subjecting all of the leachate, or a portion thereof, to one or more concentration steps comprising evaporation, which evaporation is conducted at a temperature of between about 100° C. and about 120° C. Any protein removed during the leaching may be recovered.

According to any of the above aspects of the invention, the re-circulated leachate may be supplemented with additional alkali.

According to any of the foregoing aspects of the invention, the pretreating of the leached feedstock with acid may comprise hydrolyzing at least a portion of hemicellulose present in said feedstock so as to increase accessibility of cellulose in said feedstock to being hydrolyzed with said cellulase enzymes. The pretreatment may produce sugar monomers including xylose, glucose, arabinose, mannose, galactose and a combination thereof. In one embodiment of the invention, the pretreating is conducted at a temperature of between about 160° C. to about 280° C. According to another embodiment of the invention, the pretreating is conducted for between 6 seconds and 3600 seconds. The pretreating may be conducted under a pressure of between about 50 psig and 700 psig.

The cellulase enzymes utilized in any of the foregoing aspects of the invention may comprise cellobiohydrolases (CBHs) and endoglucanases (EGs). The cellulase enzymes may further comprise beta-glucosidase.

According to a fourth aspect of the invention, there is provided a process for producing glucose from a lignocellulosic feedstock comprising the steps of:

(i) leaching the lignocellulosic feedstock with an aqueous solution to remove at least potassium salts from said lignocellulosic feedstock and without significantly hydrolyzing hemicellulose and cellulose, thereby producing a leached feedstock and leachate;
(ii) removing the leachate from leached feedstock, said leachate comprising at least potassium salt;
(iii) concentrating the leachate comprising the potassium salt to produce concentrated leachate;
(iv) pretreating the leached feedstock to produce a pretreated lignocellulosic feedstock having increased accessibility to hydrolysis of is cellulose component;
(v) adjusting the pH of the pretreated lignocellulosic feedstock by alkali addition to produce a neutralized feedstock having a pH between about 4 and about 6;
(vi) hydrolyzing cellulose in the pretreated lignocellulosic feedstock to produce glucose; and
(vii) recirculating the concentrated leachate comprising at least potassium salt to one or more stages of the process involving alkali addition to adjust the pH of a process stream.

According to a fifth aspect of the invention, there is provided a process for producing a fermentation product from a lignocellulosic feedstock comprising the steps of:

(i) leaching the lignocellulosic feedstock with an aqueous solution to remove at least potassium salts from said lignocellulosic feedstock and without significantly hydrolyzing hemicellulose and cellulose, thereby producing a leached feedstock and leachate;
(ii) removing the leachate from leached feedstock, said leachate comprising at least potassium salt;
(iii) concentrating the leachate comprising the potassium salt to produce concentrated leachate;
(iv) hydrolyzing the leached feedstock to produce fermentable sugar in one or more stages;
(v) fermenting the sugar from step (iv) to produce the fermentation product; and
(vi) recirculating the concentrated leachate comprising at least potassium salt to one or more stages of the process involving alkali addition to adjust the pH of a process stream.

The present invention can provide numerous benefits over conventional processes for converting lignocellulosic feedstock to fermentable sugar. By leaching the feedstock and removing the resultant leachate comprising potassium, the acid demand in the subsequent pretreatment can be reduced. Moreover, concentrating and then re-using the leachate at those stages in the process requiring pH adjustment by alkali addition can result in significant savings in alkali usage. Stages at which alkali usage can be reduced include, but are not limited to, after pretreatment and prior to enzymatic hydrolysis to adjust the pH of the pretreated feedstock between about 4 and about 6, prior to fermentation to adjust the pH between about 4.0 and about 6.0 or to a yeast slurry stream obtained from a yeast recycle conducted during fermentation. Reducing the cost associated with high chemical usage could be a significant step forward with respect to commercializing the process.

### BRIEF DESCRIPTION OF THE DRAWINGS

These and other features of the invention will become more apparent from the following description in which reference is made to the appended drawings wherein:

FIG. 1A depicts a block flow diagram of recycle of concentrated leachate after pretreatment and prior to cellulose hydrolysis with cellulase enzymes.

FIG. 1B depicts a block flow diagram of recycle of concentrated leachate after cellulose hydrolysis with cellulase enzymes and prior to fermentation.

FIG. 1C depicts a block flow diagram of recycle of concentrated leachate after washing the acid pretreated feedstock to obtain hemicellulose-derived sugars and before fermentation of these sugars.

FIG. 2 compares the mass of sodium hydroxide (diamonds) and the mass of sodium hydroxide following the addition of concentrated leachate (squares) to increase the pH of an acid pretreated wheat straw slurry to 4.9.

FIG. 3 shows the enzymatic hydrolysis of acid pretreated wheat straw slurry, as g/L of glucose vs. time, after pH adjustment of the pretreated slurry using concentrated leachate.

### DETAILED DESCRIPTION OF THE INVENTION

The following description is of a preferred embodiment by way of example only and without limitation to the combination of features necessary for carrying the invention into effect. The headings provided are not meant to be limiting of the various embodiments of the invention. Terms such as “comprises”, “comprising”, “comprise”, “includes”, “including” and “include” are not meant to be limiting. In addition, the use of the singular includes the plural, and “or” means “and/or” unless otherwise stated. Unless otherwise defined herein, all technical and scientific terms used herein have the same meaning as commonly understood by one of ordinary skill in the art.

### Feedstocks and Particle Size Reduction

The feedstock for the process is a lignocellulosic material. By the term “lignocellulosic feedstock”, it is meant any type of plant biomass such as, but not limited to, non-woody plant biomass, cultivated crops such as, but not limited to grasses, for example, but not limited to, C4 grasses, such as switch grass, cord grass, rye grass, miscanthus, reed canary grass, or a combination thereof, sugar processing residues, for example, but not limited to, baggase, such as sugar cane bagasse, beet pulp, or a combination thereof, agricultural residues, for example, but not limited to, soybean stover, corn stover, rice straw, sugar cane straw, rice hulls, barley straw, corn cobs, wheat straw, canola straw, oat straw, oat hulls, corn fiber, or a combination thereof, forestry biomass for example, but not limited to, recycled wood pulp fiber, sawdust, hardwood, for example aspen wood, softwood, or a combination thereof. Furthermore, the lignocellulosic feedstock may comprise cellulosic waste material or forestry waste materials such as, but not limited to, newsprint, cardboard and the like. Lignocellulosic feedstock may comprise one species of fiber or, alternatively, lignocellulosic feedstock may comprise a mixture of fibers that originate from different lignocellulosic feedstocks. In addition, the lignocellulosic feedstock may comprise fresh lignocellulosic feedstock, partially dried lignocellulosic feedstock, fully dried lignocellulosic feedstock, or a combination thereof.

Lignocellulosic feedstocks comprise cellulose in an amount greater than about 20%, more preferably greater than about 30%, more preferably greater than about 40% (w/w). For example, the lignocellulosic material may comprise from about 20% to about 50% (w/w) cellulose, or any amount therebetween. Furthermore, the lignocellulosic feedstock comprises lignin in an amount greater than about 10%, more typically in an amount greater than about 15% (w/w). The lignocellulosic feedstock may also comprise small amounts of sucrose, fructose and starch.

The feedstock typically has a pH of between about 6.0 and about 9.0 prior to leaching. The pH of the feedstock can be determined by adding water thereto (at least 10 part water to 1 part feedstock by weight) and then measuring the pH of the solution at ambient temperature. The feedstock should be subjected to size reduction to less than 2 inches prior to this pH measurement.

Lignocellulosic feedstocks of particle size less than about 6 inches may not require size reduction prior to or during leaching. That is, such feedstocks may simply be slurried in water and subjected to leaching. For feedstocks of larger particle sizes, the lignocellulosic feedstock is subjected to size reduction by methods including, but not limited to, milling, grinding, agitation, shredding, compression/expansion, or other types of mechanical action. Size reduction by mechanical action can be performed by any type of equipment adapted for the purpose, for example, but not limited to, hammer mills, tub-grinders, roll presses, refiners and hydrapulpers. At least 90% by weight of the particles produced from the size reduction may have a length less than between about 1/16 and about 6 in. The preferable equipment for the particle size reduction is a hammer mill, a refiner or a roll press as disclosed in WO 2006/026863, which is incorporated herein by reference. Before, during or subsequent to size reduction, the feedstock is typically slurried in water. This allows the feedstock to be pumped.

### Leaching of the Lignocellulosic Feedstock

The feedstock is leached prior to pretreatment to remove the inorganic salts, proteins, and other impurities out of the feedstock. By leaching the lignocellulosic feedstock, the level of compounds that increase acid demand during acid pretreatment are reduced.

The lignocellulosic feedstock contains leachable minerals, such as potassium, sodium, calcium and, in some instances, magnesium. The feedstock also contains proteins and silica.

By the term “leached feedstock”, it is meant a lignocellulosic feedstock that has been in contact with an aqueous solution to remove at least potassium. In one exemplary embodiment of the invention, at least 75% of the potassium is removed from the feedstock during leaching. In another embodiment of the invention, at least 80% of the potassium, or at least 85% of the potassium is removed from the lignocellulosic feedstock during leaching. This includes all ranges therebetween, such as ranges containing numerical limits of 75, 80, 85, 90, 95 or 100%.

Optionally, sodium, a portion of calcium and a portion of magnesium, if present in the feedstock, are removed as well. Protein is soluble and thus can be removed by leaching. Silica may be removed as well, although it has little to no effect on acid or alkali requirements added in downstream stages of the process.

The pH, temperature and duration of the leaching are selected so that limited hydrolysis of the hemicellulose and cellulose in the feedstock occurs. A person of ordinary skill in the art would be aware of the inter-dependence of these variables and could adjust them as required.

Leaching is conducted “without significantly hydrolyzing hemicellulose and cellulose”. In this context, “without significantly hydrolyzing”, means that less than 5 wt % of the hemicellulose and cellulose is hydrolyzed to oligomers, sugar monomers, or a combination thereof. Preferably less than 2 wt % of the hemicellulose and cellulose is hydrolyzed.

Leaching may be preceded by a mild steam conditioning step. In such a step, the feedstock is subjected to low pressure steam at a temperature of between about 50° C. and 90° C., more typically between 70° C. and 85° C. This includes all ranges therebetween, such as ranges containing numerical limits of 50, 55, 60, 65, 70, 75, 80, 85 or 90° C.

Acetyl groups present on the lignocellulosic feedstock will typically remain largely intact during the leaching step. However, as discussed below, the leaching may be followed by an alkaline conditioning step, the purpose of which is to remove acetyl groups.

Leaching may comprise contacting lignocellulosic feedstock with an aqueous solution for a period between about 2 minutes and about 5 hours, between about 2 minutes and about 4 hours, between about 2 minutes and about 3 hours, between about 2 minutes and about 2 hours or between about 10 minutes and about 30 minutes. This includes all ranges therebetween, such as ranges containing numerical limits of 2 minutes, 10 minutes, 30 minutes, 1 hour, 2 hours, 3 hours, 4 hours or 5 hours.

Leaching may be performed at a temperature between about 4° C. and about 95° C. or between about 20° C. and about 80° C., or between about 20° C. and about 60° C. In exemplary embodiments of the invention, leaching is performed within ranges having numerical limits of about 20° C., 25° C., 30° C., 35° C., 40° C., 45° C., 50° C., 55° C., 60° C., 65° C., 70° C., 75° C., 80° C., 85° C., 90° C. or 95° C. Alternatively, the leaching may be performed at higher temperatures than this and under pressure, for example at temperatures greater than 95° C.

The aqueous solution used to leach the feedstock may have a pH between about 6 and about 9. More acidic solutions used to leach the feedstock will remove diavalent cations, such as calcium and magnesium. The aqueous solution used for leaching may be water, process water, fresh water, or a combination thereof. On the other hand, solutions that are mildly acidic, neutral or mildly alkaline may leave most or all of the calcium and magnesium in the feedstock intact, but remove all or a majority of the potassium and sodium from the feedstock. The pH of the aqueous solution may be adjusted using small amounts of any suitable alkali, such as sodium hydroxide. An alkaline solution could be advantageous for protein extraction.

Without being limiting the pH of the aqueous solution used to leach the lignocellulosic feedstock may be within a range having numerical limits of about 6.0, 6.5, 7.0, 7.5, 8.0, 8.5 or 9.0.

In one embodiment of the invention, the aqueous solution employed in leaching the lignocellulosic feedstock comprises about 0.25 to about 10 times the maximum water holding capacity per kilogram of dry lignocellulosic feedstock, or about 1.5 to about 3 times the maximum water holding capacity per kilogram of dry lignocellulosic feedstock. The leaching may be performed using an aqueous solution comprising more than about 10 times the maximum water holding capacity per kilogram of lignocellulosic feedstock. The maximum water holding capacity of a lignocellulosic feedstock may be determined by, for example, measuring the volume of water which may be absorbed by a known mass of loosely packed lignocellulosic feedstock until the point at which additional water added to the feedstock is free water. This point may be estimated as the point wherein water forms a thin continuous layer of the lignocellulosic feedstock. In determining the maximum water holding capacity of a feedstock, it is preferable that the lignocellulosic feedstock is mechanically disrupted into particles of about the same size. Further, as would be evident to a person skilled in the art, it is preferred that the maximum water holding capacity of a feedstock be determined on a loosely packed and not tightly packed lignocellulosic feedstock.

Leachate may be removed from the leached feedstock by any suitable solids-liquid separation such as pressing, washing, centrifugation, microfiltration, plate and frame filtration, crossflow filtration, pressure filtration, vacuum filtration and the like. As would be evident to those of skill in the art, the step of removing leachate from the leached feedstock need not result in complete removal of all aqueous solution from the leached feedstock.

The leaching step may be a batch or a continuous process. If the leaching is a continuous operation, it may be conducted co-current or counter-current with respect to the point of addition and withdrawal of the leachate from the feedstock.

In one exemplary embodiment of the invention, the leaching contains multiple stages with co-current and/or counter-current contact of liquids and solids. Each stage of the leaching operation will typically include a separation step where the leachate is removed from the leached or partially-leached lignocellulosic feedstock. Leachate removed from one or more of the leaching stages may be re-used in other leaching stages. As discussed previously, the leachate may be added back to one or more leaching stages of the process so that it contacts the feedstock solids in a co-current or counter-current fashion.

The leaching of the present invention may comprise a leaching bath where the feedstock remains submerged for a predetermined amount of time. This step may be conducted in a tank adapted for removal of sand particles and other heavy debris that may settle to the bottom of the tank. The settled sand and other debris may be subsequently conveyed out of the tank and discarded.

As mentioned previously, the leachate removed from the lignocellulosic feedstock during or after leaching will comprise at least potassium. Depending on the leaching conditions, the leachate may also contain some calcium. Magnesium and sodium may be removed as well if the feedstock contains salts of these cations.

As mentioned previously, leaching can also recover protein, which may be useful as an animal feed. The choice of temperature for the leaching process may therefore involve a balance between the higher leaching efficiency at high temperatures and the protein stability at lower temperatures. The leachate may also comprise undissolved substances, such as fine particles of the lignocellulosic feedstock.

### Concentration of Leachate

All or a portion of the leachate is concentrated after it is removed from the lignocellulosic feedstock. That which is not concentrated may be disposed of as a bleed stream from the process. Typically, the leachate will have a concentration of between about 1 to 10 wt % total dissolved solids, more typically about 3 to about 5 wt % (w/w). For example, the leachate may have a concentration of 1, 2, 3, 4, 5, 6, 7, 8, 9 or 10 wt % total dissolved solids. The leachate can be concentrated by any suitable technique known to those of ordinary skill in the art. Non-limiting examples of suitable concentration methods include evaporation or reverse osmosis.

The evaporation may be conducted using any suitable evaporator known to those of skill in the art. Evaporators used in industry contain a heating section, a concentrating and separating section and a vacuum or pump to provide pressure. The most common heating section used in industry consists of parallel tubes, although plates and coils may be used as well.

Concentration of the leachate may be carried out with a falling film evaporator. Such evaporators are generally made of bundles of long tubes that are surrounded by a steam jacket. Liquid flows downward in each tube, forming a thin film on the inside wall, while steam condenses and flows downward on the outer surface of the tube. Boiling/evaporation take place in the thin film because of the heat applied by the steam. The vapour produced by this boiling/evaporation and the liquid concentrated by the process flow downward. The vapour leaves the top of the evaporator, while the concentrated liquid is discharged from the bottom of the unit.

The evaporation may be carried out in a single-stage evaporator or may be a multiple-effect system, i.e., a system in which more than one evaporator is employed. The evaporation is typically a continuous process.

Multiple-effect evaporator systems provide for optimal steam economy, but have the drawback of increased capital expenditure relative to single effect evaporators. A single effect evaporator uses more steam than a multiple-effect system during operation, but requires less capital investment. A person of skill in the art can readily choose a suitable evaporation system by taking into account the foregoing cost considerations.

A multiple-effect evaporator system utilized in accordance with the invention can be forward fed, meaning that the feeding takes place so that the solution to be concentrated enters the system through the first effect, which is at the highest temperature. Partial concentration occurs in the first effect, with vapour sent to the second effect to provide heat for same. The partially concentrated leachate solution is then sent to the second effect where it is again partially concentrated, with vapour sent to the third effect, and so on. Alternatively, backward feeding may be utilized, in which the partially concentrated solution is fed from effect to effect with increasing temperature.

Falling film evaporation will typically concentrate the leachate to 55-65% (w/w) dissolved solids. To achieve higher concentrations than this, other types of evaporation units can be employed. This includes, but is not limited to, forced re-circulation evaporators and mechanical vapour recompression units. Further concentration can be employed to increase the undissolved solids concentration to 75% (w/w) or higher.

Mechanical vapor recompression (MVR) systems are equipped with one or more compressors to increase the pressure of the vapour stream. This increases the condensation temperature of the vapour. With the vapour at a higher temperature, it can then be used to provide energy to the system. Typically MVR evaporators are arranged in parallel.

Forced circulation evaporators employ a pump to increase pressure and circulation. This can avoid drying out of the system that can occur when using such evaporators. Forced recirculation evaporators may include a wiper blade for concentrating solutions containing solids or that have fouling tendencies.

A person of skill in the art can readily select a suitable operating temperature for the evaporation. In one embodiment of the invention, the operating temperature is between about 70° C. and about 140° C. to aid decomposition of potassium bicarbonate to potassium carbonate, carbon dioxide and water.

The pressure employed during evaporation will typically vary between 1.4×10⁵ and 2.0×10⁵ pascal. Higher pressure could potentially be employed, but will require registered pressure vessels, which increases cost.

The vacuum applied to the system can be as low as 0.4×10⁵ pascal.

A reverse osmosis unit can be utilized prior to evaporation to pre-concentrate the leachate, depending on the osmotic pressure of the solution.

A suitable metallurgy for use in the evaporators can readily be selected by those of ordinary skill in the art and depends on such considerations as pH, temperature and the chemical composition of the leachate. For example, high chloride concentrations will typically require the use of molybdenum on the surfaces of the evaporators exposed to the leachate. In those embodiments employing multiple-effect evaporators, the metallurgy may be the same in all the evaporators in the system or can differ among the effects.

Re-use of the condensated steam from the evaporation may be carried out depending upon whether organics are present in the condensate. If organic compounds are present, the condensate will typically be diverted to wastewater treatment. Condensed steam may be re-circulated to a boiler or to other stages of the process.

A further example of a technique for concentrating the leachate is membrane filtration. Membrane filtration is a process of filtering a solution with a membrane so as to concentrate it. This includes microfiltration, which employs membranes of a pore size of 0.05-1 microns for the removal of particulate matter; ultrafiltration, which employs membranes with a cut-off of 500-50,000 mw for removing large soluble molecules; and reverse osmosis using nanofiltration membranes to separate small molecules from water. Membrane filtration may be used for clarification as well as concentration. Clarification is generally carried out prior to those filtration techniques utilizing smaller pore sizes, such as reverse osmosis to prevent fouling of the membrane. Two or more membrane filtrations could be utilized as required.

In one example of the invention, the leachate is concentrated by reverse osmosis. As would be appreciated by those of skill in the art, reverse osmosis involves the separation of solutions having different solute concentrations with a semi-permeable membrane by applying sufficient pressure to a liquid having a higher solute concentration to reverse the direction of osmosis across the membrane.

In the practice of the invention, the semi-permeable membrane does not allow the potassium salts and other salts in the leachate to move from one compartment to the other, but allows water to pass freely. Sufficient pressure is applied to the side of the membrane having the higher potassium salt concentration so that the osmotic pressure across the membrane is overcome. This allows the passage of water from the solution containing the higher concentration of potassium salt to the solution containing the lower concentration of the salt. An example of a suitable pressure is greater than 500 psig.

Membranes used for reverse osmosis generally have a dense barrier layer in the polymer matrix where most separation occurs. The semi-permeable membranes for reverse osmosis treatment are generally constructed from polyamide-based materials. Reverse osmosis membranes are made in a variety of configurations, with the most common being spiral-wound and hollow-fiber. As will be appreciated by those of ordinary skill in the art, the pressure exerted on the membrane will depend on the nature of the solute to be concentrated.

The leachate may need to be pre-treated prior to reverse osmosis. If the concentration of the calcium or magnesium salts in the leachate is at a level high enough that the salts are insoluble, it will create a hard mineral on the inside of the membrane, rendering it ineffective.

Undissolved substances within the leachate, such as, but not limited to, fine particles of the lignocellulosic feedstock are preferably removed to prevent fouling of the reverse osmosis membrane and reduce the risk of damage to high-pressure pump components. Oxidizing biocides may be added to the leachate to prevent bacterial growth on the membrane surface. Biofouling inhibitors may also be added to the membranes to prevent bacterial growth. Other pretreatment methods to prevent fouling include cartridge filtration, which involves passage through string-wound polypropylene filters that remove between 1-5 micrometer sized particles, pH adjustment to prevent scale and the addition of scale inhibitors.

The final solids concentration of the concentrated leachate may be between about 20 wt % and about 80 wt % measured as total solids, more typically between about 50 wt % and about 75 wt %. In embodiments of the invention, the final solids content is any range therebetween, for example having numerical limits of about 20, 25, 30, 35, 40, 45, 50, 55, 60, 65, 70, 75 or 80 wt %.

The pH of the concentrated leachate will be between about 7.0 to about 12.0. In embodiments of the invention, the pH of the concentrated leachate is between about 9.0 and about 12.0. This includes any sub-range therebetween, including ranges having numerical limits of 7.0, 7.5, 8.0, 8.5, 9.0, 9.5, 10.0, 10.5, 11.0, 11.5 or 12.0.

The concentrated leachate is recirculated to “one or more stage of the process involving alkali addition”. By this it is meant any stage or stages of the process in which alkali is added to a process stream to adjust its pH, including, but not limited to an acid pretreated lignocellulosic feedstock prior to enzymatic hydrolysis; a stream comprising glucose prior to its fermentation; a stream comprising xylose prior to its fermentation and/or a yeast slurry stream. Each of these process streams are described in more detail hereinafter.

It should be appreciated that other alkali, such as sodium hydroxide, potassium hydroxide, ammonia or ammonium hydroxide, may also be added to the leachate to increase its pH prior to its recirculation. The alkali can be mixed with the concentrated leachate and then recirculated, or the two bases can be added separately. Mixing of the alkali prior to their addition is advantageous as it necessitates only one alkali addition point. However, before mixing, caution should be taken to ensure that the two solutions are chemically compatible.

If the concentrated leachate is supplemented with ammonia, it may be added directly to the slurry as ammonia gas. Alternatively, the gas may be pre-dissolved in water to form an ammonium hydroxide solution, which can then be added to the concentrated leachate.

### Dilute Acid Pretreatment of the Lignocellulosic Feedstock

Subsequent to leaching, the leached feedstock is subjected to pretreatment, typically with a mineral acid. The acid pretreatment is intended to deliver a sufficient combination of mechanical and chemical action so as to disrupt the fiber structure of the lignocellulosic feedstock and increase the surface area of the feedstock to make it accessible to cellulase enzymes. Preferably, the acid pretreatment is performed so that nearly complete hydrolysis of the hemicellulose and only a small amount of conversion of cellulose to glucose occurs. The cellulose is hydrolyzed to glucose in a subsequent step that uses cellulase enzymes. Generally, a dilute mineral acid, at a concentration from about 0.02% (w/w) to about 5% (w/w), or any amount therebetween, (measured as the percentage weight of pure acid in the total weight of dry feedstock plus aqueous solution) is used for the pretreatment.

The acid may be sulfuric acid, sulfurous acid, hydrochloric acid or phosphoric acid. Preferably, the acid is sulfuric acid. The amount of acid added to the lignocellulosic feedstock may vary, but should be sufficient to achieve a final concentration of acid of about 0.02 wt % to about 2 wt %, or any amount therebetween. The resulting pH of the feedstock is about pH 0.4 to about pH 3.5, or any pH range therebetween. For example, the pH of the slurry may be between about 0.4, 1.0, 1.5, 2.0, 2.5, 3.0 or 3.5.

The acid pretreatment is preferably carried out at a maximum temperature of about 160° C. to about 280° C. However, it should be understood that, in practice, there will be a time delay in the pretreatment process before the feedstock reaches this temperature range. Thus, the above temperatures correspond to those values reached after sufficient application of heat to reach a temperature within this range. The time that the feedstock is held at this temperature may be about 6 seconds to about 3600 seconds, or about 15 seconds to about 750 seconds or about 30 seconds to about 240 seconds.

The feedstock may be heated with steam during pretreatment. Without being limiting, one method to carry this out is to use low pressure steam to partially heat the feedstock, which is then pumped to a heating train of several stages.

The pretreatment may be carried out under pressure. For example, the pressure during pretreatment may be between about 50 and about 700 psig or between about 75 and about 600 psig, or any pressure range therebetween. That is, the pretreatment may be carried out at 50, 100, 75, 150, 200, 250, 300, 350, 400, 450, 500, 550, 600, 650 or 700 psig, or any pressure therebetween.

The pretreatment is generally carried out at a solids consistency of 5% to 30% (w/w). The solids consistency is measured by drying at 105° C. overnight, as familiar to those skilled in the art. Those skilled in the art are aware that a solids consistency below this range introduces excess water into the system, while a solids consistency above this range is generally too difficult to pump.

One method of performing acid pretreatment of the feedstock is steam explosion using the process conditions set out in U.S. Pat. No. 4,461,648 (Foody, which is herein incorporated by reference). Another method of pretreating the feedstock slurry involves continuous pretreatment, meaning that the lignocellulosic feedstock is pumped through a reactor continuously. Continuous acid pretreatment is familiar to those skilled in the art; see, for example, U.S. Pat. No. 5,536,325 (Brink); WO 2006/128304 (Foody and Tolan); and U.S. Pat. No. 4,237,226 (Grethlein), which are each incorporated herein by reference. Additional techniques known in the art may be used as required such as the process disclosed in U.S. Pat. No. 4,556,430 (Converse et al.; which is incorporated herein by reference).

The pH of the pretreatment is measured by removing a sample from the pretreatment process after acid addition and measuring the pH of the sample, as is familiar to those of ordinary skill in the art.

The acid pretreatment produces a composition comprising an acid pretreated feedstock. Sugars produced by the hydrolysis of hemicellulose during pretreatment are generally present in the composition and include xylose, glucose, arabinose, mannose, galactose or a combination thereof.

The aqueous phase of the composition comprising the pretreated feedstock may also contain the acid added during the pretreatment. When sulfuric acid is the acid utilized in the pretreatment, the composition comprising the pretreated feedstock additionally contains sulfate and/or bisulfate salts of potassium, sodium, calcium and possibly magnesium. These salts include potassium sulfate, potassium bisulfate, sodium sulfate, sodium bisulfate, calcium sulfate and magnesium sulfate.

As discussed previously, the composition comprising acid pretreated feedstock will also comprise acetic acid produced during acid pretreatment. The concentration of acetic acid in this stream may be between 0.1 and 20 g/L. Additional organic acids may be liberated during pretreatment, including galacturonic acid, formic acid, lactic acid and glucuronic acid. Pretreatment may also produce dissolved lignin and inhibitors such as furfural and hydroxymethyl furfural (HMF). Accordingly, the composition comprising acid pretreated feedstock may also contain these components.

According to one exemplary embodiment of the invention, the soluble components of the pretreated feedstock composition are separated from the solids. This separation may be carried out by washing the pretreated feedstock composition with an aqueous solution to produce a wash stream, and a solids stream comprising the unhydrolyzed, pretreated feedstock. Alternatively, the soluble component is separated from the solids by subjecting the pretreated feedstock composition to a solids-liquid separation, using known methods such as centrifugation, microfiltration, plate and frame filtration, cross-flow filtration, pressure filtration, vacuum filtration and the like. Optionally, a washing step may be incorporated into the solids-liquids separation. The separated solids, which contain cellulose, may then be sent to enzymatic hydrolysis with cellulase enzymes in order to convert the cellulose to glucose. The enzymatic hydrolysis of cellulose using cellulase enzymes is described in more detail hereinafter.

The aqueous stream, which includes the sugars released during pretreatment, the pretreatment acid and other soluble components, may then be fermented using a microorganism capable of fermenting the sugars derived from the hemicellulose component of the feedstock. Examples of suitable microorganisms for fermenting such sugars are described hereinafter.

The pH of the aqueous stream separated from the feedstock solids will be acidic after the acid pretreatment. Since most fermentations are conducted within a pH range of between about 4.0 and about 6.0, the pH of the pretreated feedstock will need to be increased. This includes ranges therebetween having numerical limits of 4.0, 4.25, 4.5, 4.75, 5.0, 5.25, 5.5, 5.75 or 6.0. In one embodiment of the invention, the pH of the aqueous stream is adjusted by the addition of the concentrated leachate. It should be appreciated that other alkali, such as sodium hydroxide, potassium hydroxide, ammonia or ammonium hydroxide, may also be added to the aqueous stream to adjust its pH.

### Enzymatic Hydrolysis

The enzymatic hydrolysis is conducted at a pH between about 4.0 and 6.0 as this is within the optimal pH range of most cellulases. Since the pH of the pretreated lignocellulosic feedstock is acidic, its pH will need to be increased to about pH 4.0 to about 6.0 prior to enzymatic hydrolysis, or more typically between about 4.5 and about 5.5. This includes ranges therebetween having numerical limits of 4.0, 4.25, 4.5, 4.75, 5.0, 5.25, 5.5, 5.75 or 6.0. However, cellulases with pH optima at more acidic and more alkaline pH values are known.

The concentrated leachate can be added to the pretreated feedstock after it is cooled, before cooling, or at points both before and after cooling. Such a cooling step is typically carried out prior to enzymatic hydrolysis to cool the pretreated feedstock to a temperature suitable for the cellulase enzymes. It should be appreciated that cooling of the feedstock can occur in a number of stages and the point(s) of the concentrated leachate addition need not be after cooling to a temperature suitable for enzymatic hydrolysis. Cooling of the pretreated feedstock may be carried out by flashing, heat exchange or other suitable means.

In one embodiment of the invention, the pretreated feedstock is cooled to temperatures of about 100° C. and below. For example, the temperature of the pretreated feedstock upon addition of the concentrated leachate may vary between about 50° C. and about 120° C. or more typically between about 50° C. and about 100° C. This range includes temperatures typical of pretreatment and temperatures that would be utilized in enzymatic hydrolysis.

Decomposition of potassium bicarbonate occurs between 100° C. and 120° C. into K₂CO₃, H₂O and CO₂. Thus, if the leachate comprises significant levels of potassium bicarbonate, and the pH adjustment is conducted within the temperature range at which thermal decomposition occurs, by driving off CO₂, the stream becomes more alkaline. However, the gassing off of the CO₂ then has to be managed.

The concentrated leachate may be added in-line to the pretreated feedstock, such as an in-line mixer, to a pump downstream of pretreatment, directly to a hydrolysis vessel and/or to a hydrolysis make-up tank employed to mix the cellulase enzymes and concentrated leachate prior to addition of the mixture to an enzymatic hydrolysis reactor.

The concentrated leachate could also potentially be added to one or more flash vessel downstream of pretreatment to ensure good mixing of the feedstock, although it will typically be most advantageous to avoid addition of the leachate at this point as it can have negative consequences on downstream processing.

If the concentrated leachate is added to a make-up tank prior to enzyme addition, the residence time in the tank may be between about 0.5 and about 30 minutes. The hydrolysis make-up vessel volume may be between about 1000 and about 50,000 liters and feed and withdrawal of the vessel contents may be batch, fed-batch or continuous.

Alternatively, the pretreated feedstock is fed to a hydrolysis make-up tank, along with cellulase enzymes and concentrated leachate to adjust its pH. The contents of the make-up tank will typically be mixed and then submitted to a hydrolysis reactor.

It should be appreciated that other alkali, such as sodium hydroxide, potassium hydroxide, ammonia or ammonium hydroxide, may also be added to the aqueous stream to increase its pII. The alkali can be mixed with the concentrated leachate and then added to the pretreated feedstock, or the two bases can be added separately. Mixing of the alkali prior to their addition is advantageous as it necessitates only one alkali addition point. However, before mixing, caution should be taken to ensure that the two solutions are chemically compatible.

If the concentrated leachate is supplemented with ammonia, it may be added directly to the slurry as ammonia gas. Alternatively, the gas may be pre-dissolved in water to form an ammonium hydroxide solution, which can then be added to the concentrated leachate.

The fiber solids concentration of the pretreated lignocellulosic feedstock to which the leachate is added, and optionally other alkali, may be between about 1% and about 25% (w/v) or between about 8% and about 18% (w/v). This includes all fiber solids concentrations therebetween, including ranges having limits of 1, 5, 10, 15, 20 or 25% (w/v). As is well known to those of ordinary skill in the art, the concentration of suspended or undissolved solids can be determined by filtering a sample of a feedstock slurry using glass microfiber filter paper, washing the filter cake with water and drying the cake overnight at 105° C.

The enzymatic hydrolysis can be carried out with any type of cellulase enzymes suitable for such purpose and effective at the pH and other conditions utilized, regardless of their source. Among the most widely studied, characterized and commercially produced cellulases are those obtained from fungi of the genera Aspergillus, Humicola, Chrysosporium, Melanocarpus, Myceliopthora and Trichoderma, and from the bacteria of the genera Bacillus and Thermobifida. Cellulase produced by the filamentous fungi Trichoderma longibrachiatum comprises at least two cellobiohydrolase enzymes termed CBHI and CBHII and at least four EG enzymes. As well, EGI, EGII, EGIII, EG V and EGVI cellulases have been isolated from Humicola insolens (see Lynd et al., 2002, Microbiology and Molecular Biology Reviews, 66(3):506-577 for a review of cellulase enzyme systems and Coutinho and Henrissat, 1999, “Carbohydrate-active enzymes: an integrated database approach.” In Recent Advances in Carbohydrate Bioengineering, Gilbert, Davies, Henrissat and Svensson eds., The Royal Society of Chemistry, Cambridge, pp. 3-12, each of which are incorporated herein by reference).

An appropriate cellulase dosage can be about 1.0 to about 40.0 Filter Paper Units (FPU or IU) per gram of cellulose, or any amount therebetween. The FPU is a standard measurement familiar to those skilled in the art and is defined and measured according to Ghose (Pure and Appl. Chem., 1987, 59:257-268; which is incorporated herein by reference). A preferred cellulase dosage is about 10 to 20 FPU per gram cellulose.

The conversion of cellobiose to glucose is carried out by the enzyme β-glucosidase. By the term “β-glucosidase”, it is meant any enzyme that hydrolyzes the glucose dimer, cellobiose, to glucose. The activity of the β-glucosidase enzyme is defined by its activity by the Enzyme Commission as EC#3.2.1.21. The β-glucosidase enzyme may come from various sources; however, in all cases, the β-glucosidase enzyme can hydrolyze cellobiose to glucose. The ⊖-glucosidase enzyme may be a Family 1 or Family 3 glycoside hydrolase, although other family members may be used in the practice of this invention. The preferred β-glucosidase enzyme for use in this invention is the Bgl1 protein from Trichoderma reesei. It is also contemplated that the β-glucosidase enzyme may be modified to include a cellulose binding domain, thereby allowing this enzyme to bind to cellulose.

The cellulase enzymes and β-glucosidase enzymes may be handled in an aqueous solution or as a powder or granulate. The enzymes may be added to the pretreated feedstock at any point prior to its introduction into a hydrolysis reactor. Alternatively, the enzymes may be added directly to the hydrolysis reactor, although the addition of enzymes prior to the introduction of the pretreated feedstock into the hydrolysis reactor is preferred for optimal mixing. The enzymes may be mixed into the pretreated feedstock using mixing equipment that is familiar to those of skill in the art.

In practice, the hydrolysis is carried out in a hydrolysis system, which includes multiple hydrolysis reactors. The number of hydrolysis reactors in the system depends on the cost of the reactors, the volume of the aqueous slurry, and other factors. For a commercial-scale ethanol plant, the typical number of hydrolysis reactors may be for example, 3 to 12. In order to maintain the desired hydrolysis temperature, the hydrolysis reactors may be jacketed with steam, hot water, or other heat sources. Preferably, the cellulase hydrolysis is a continuous process, with continuous feeding of pretreated lignocellulosic feedstock and withdrawal of the hydrolyzate slurry. However, it should be understood that batch and fed-batch processes are also included within the scope of the present invention.

Other design parameters of the hydrolysis system may be adjusted as required. For example, the volume of a hydrolysis reactor in a cellulose hydrolysis system can range from about 100,000 L to about 20,000,000 L, or any volume therebetwecn, for example, between 200,000 and 5,000,000 L, or any amount therebetween. The total residence time of the slurry in a hydrolysis system may be between about 12 hours to about 200 hours, or any amount therebetween. The hydrolysis reactors may be unmixed or subjected to light agitation, typically with a maximum power input of up to 0.8 hp/1000 gallons, or may receive heavy agitation of up to 20 hp/1000 gallons.

Following enzymatic hydrolysis of the pretreated feedstock, any insoluble solids present in the resulting sugar stream, including lignin, may be removed using conventional solid-liquid separation techniques prior to any further processing. However, it may be desirable in some circumstances to carry forward both the solids and liquids in the sugar stream for further processing.

### Fermentation

In accordance with the invention, the fermentation is conducted at a pH between about 4.0 and about 6.0, or between about 4.5 and about 6.0. This includes all subranges and values therebetween, including ranges having pH values of 4.0, 4.5, 5.0, 5.5 or 6.0. Fermentation of glucose resulting from cellulose hydrolysis may produce one or more of the fermentation products selected from an alcohol, a sugar alcohol, an organic acid and a combination thereof. Pentose sugars arising from the hemicellulose component of the feedstock may be fermented as well. These hemicellulose sugars may be present in the stream comprising glucose, depending upon whether they separated after acid pretreatment.

To attain the foregoing pH range for fermentation, it may be necessary to add alkali to the stream comprising glucose. If the pH of this stream needs to be increased, the concentrated leachate may be re-circulated to this stage of the process to reduce alkali requirements. Similar to the other alkali addition points in the process, other alkali may be introduced as well to increase the pH of the glucose stream. This includes ammonium hydroxide, ammonia, potassium hydroxide and sodium hydroxide.

During fermentation, yeast recycle may be employed. This involves separating yeast from the fermentation broth to produce a yeast slurry stream and a liquid stream and then re-circulating the yeast slurry to the fermentor, while the liquid stream is sent to distillation. Acid may be added to the yeast slurry to lower the pH of the slurry in order to reduce the concentration of any microbial contaminants. Subsequently, alkali may be added to the yeast slurry to increase the pH of the yeast slurry prior to its re-introduction to the fermentor.

In one embodiment of the invention, the fermentation product is an alcohol, such as ethanol or butanol. For ethanol production, fermentation is typically carried out with a Saccharomyces spp. yeast. Glucose and any other hexoses present in the sugar stream may be fermented to ethanol by wild-type Saccharomyces cerevisiae, although genetically modified yeasts may be employed as well, as discussed below. The ethanol may then be distilled to obtain a concentrated ethanol solution. Butanol may be produced from glucose by a microorganism such as Clostridium acetobutylicum and then concentrated by distillation.

As mentioned previously, in addition to the glucose resulting from enzymatic hydrolysis, sugars liberated during pretreatment, namely xylose, arabinose, mannose, galactose, or a combination thereof, will be typically also present in the stream sent to fermentation.

Xylose and arabinose may also be fermented to ethanol by a yeast strain that naturally contains, or has been engineered to contain, the ability to ferment these sugars to ethanol. Examples of microbes that have been genetically modified to ferment xylose include recombinant Saccharomyces strains into which has been inserted either (a) the xylose reductase (XR) and xylitol dehydrogenase (XDH) genes from Pichia stipitis (U.S. Pat. Nos. 5,789,210, 5,866,382, 6,582,944 and 7,527,927 and European Patent No. 450530) or (b) fungal or bacterial xylose isomerase (XI) gene (U.S. Pat. Nos. 6,475,768 and 7,622,284). Examples of yeasts that have been genetically modified to ferment L-arabinose include, but are not limited to, recombinant Saccharomyces strains into which genes from either fungal (U.S. Pat. No. 7,527,951) or bacterial (WO 2008/041840) arabinose metabolic pathways have been inserted.

Organic acids that may be produced during the fermentation include lactic acid, citric acid, ascorbic acid, malic acid, succinic acid, pyruvic acid, hydroxypropanoic acid, itaconoic acid and acetic acid. In a non-limiting example, lactic acid is the fermentation product of interest. The most well-known industrial microorganisms for lactic acid production from glucose are species of the genera Lactobacillus, Bacillus and Rhizopus.

Moreover, xylose and other pentose sugars may be fermented to xylitol by yeast strains selected from the group consisting of Candida, Pichia, Pachysolen, Hansenula, Debaryomyces, Kluyveromyces and Saccharomyces. Bacteria are also known to produce xylitol, including Corynebacterium sp., Enterobacter liquefaciens and Mycobacterium smegmatis.

In practice, the fermentation is typically performed at or near the temperature and pH optimum of the fermentation microorganism. A typical temperature range for the fermentation of glucose to ethanol using Saccharomyces cerevisiae is between about 25° C. and about 35° C., although the temperature may be higher if the yeast is naturally or genetically modified to be thermostable. The dose of the fermentation microorganism will depend on other factors, such as the activity of the fermentation microorganism, the desired fermentation time, the volume of the reactor and other parameters. It should be appreciated that these parameters may be adjusted as desired by one of skill in the art to achieve optimal fermentation conditions.

The fermentation may also be supplemented with additional nutrients required for the growth of the fermentation microorganism. For example, yeast extract, specific amino acids, phosphate, nitrogen sources, salts, trace elements and vitamins may be added to the hydrolyzate slurry to support their growth.

The fermentation may be conducted in batch, continuous or fed-batch modes with or without agitation. Preferably, the fermentation reactors are agitated lightly with mechanical agitation. A typical, commercial-scale fermentation may be conducted using multiple reactors. The fermentation microorganisms may be recycled back to the fermentor or may be sent to distillation without recycle.

If ethanol or butanol is the fermentation product, the recovery is carried out by distillation, typically with further concentration by molecular sieves or membrane extraction.

The fermentation broth that is sent to distillation is a dilute alcohol solution containing solids, including unconverted cellulose, and any components added during the fermentation to support growth of the microorganisms.

Microorganisms are potentially present during the distillation depending upon whether or not they are recycled during the fermentation. The broth is preferably degassed to remove carbon dioxide and then pumped through one or more distillation columns to separate the alcohol from the other components in the broth. The mode of operation of the distillation system depends on whether the alcohol has a lower or a higher boiling point than water. Most often, the alcohol has a lower boiling point than water, as is the case when ethanol is distilled.

In those embodiments where ethanol is concentrated, the column(s) in the distillation unit is preferably operated in a continuous mode, although it should be understood that batch processes are also encompassed by the present invention. Heat for the distillation process may be introduced at one or more points either by direct steam injection or indirectly via heat exchangers. The distillation unit may contain one or more separate beer and rectifying columns, in which case dilute beer is sent to the beer column where it is partially concentrated. From the beer column, the vapour goes to a rectification column for further purification. Alternatively, a distillation column is employed that comprises an integral enriching or rectification section.

After distillation, the water remaining may be removed from the vapour by a molecular sieve resin, by membrane extraction, or other methods known to those of skill in the art for concentration of ethanol beyond the 95% that is typically achieved by distillation. The vapour may then be condensed and denatured.

An aqueous stream(s) remaining after ethanol distillation and containing solids, referred to herein as “still bottoms”, is withdrawn from the bottom of one or more of the column(s) of the distillation unit. This stream will contain inorganic salts, unfermented sugars and organic salts.

When the alcohol has a higher boiling point than water, such as butanol, the distillation is run to remove the water and other volatile compounds from the alcohol. The water vapor exits the top of the distillation column and is known as the “overhead stream”.

### EXAMPLES

### Example 1

### Recycle of Leachate to Alkali Addition Steps Conducted Prior to Enzymatic Hydrolysis or Fermentation

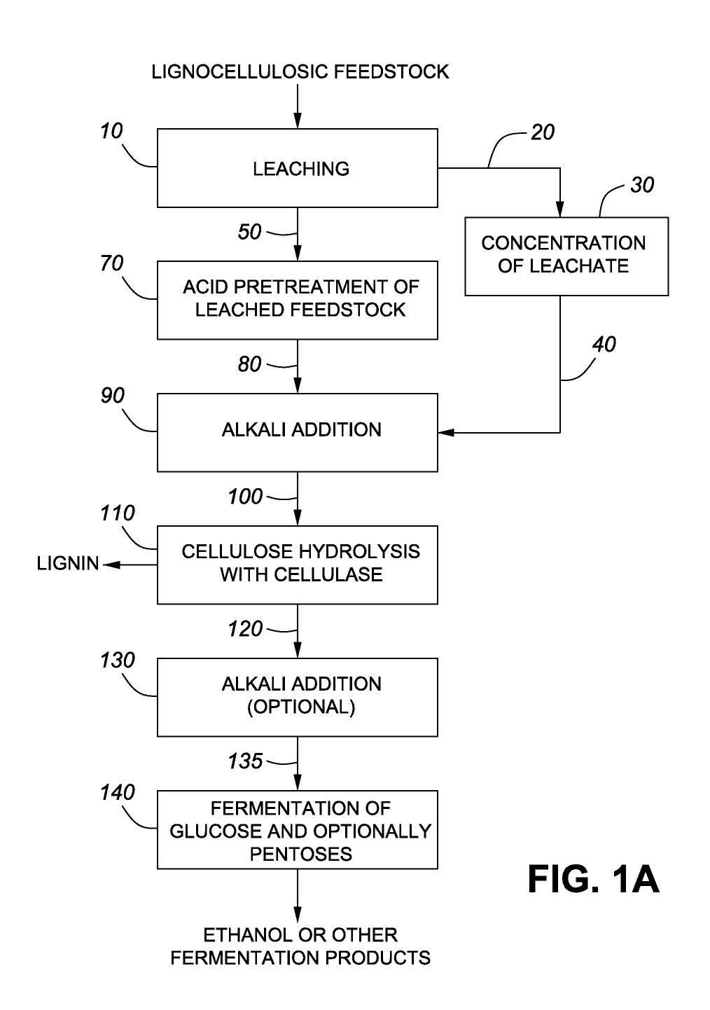

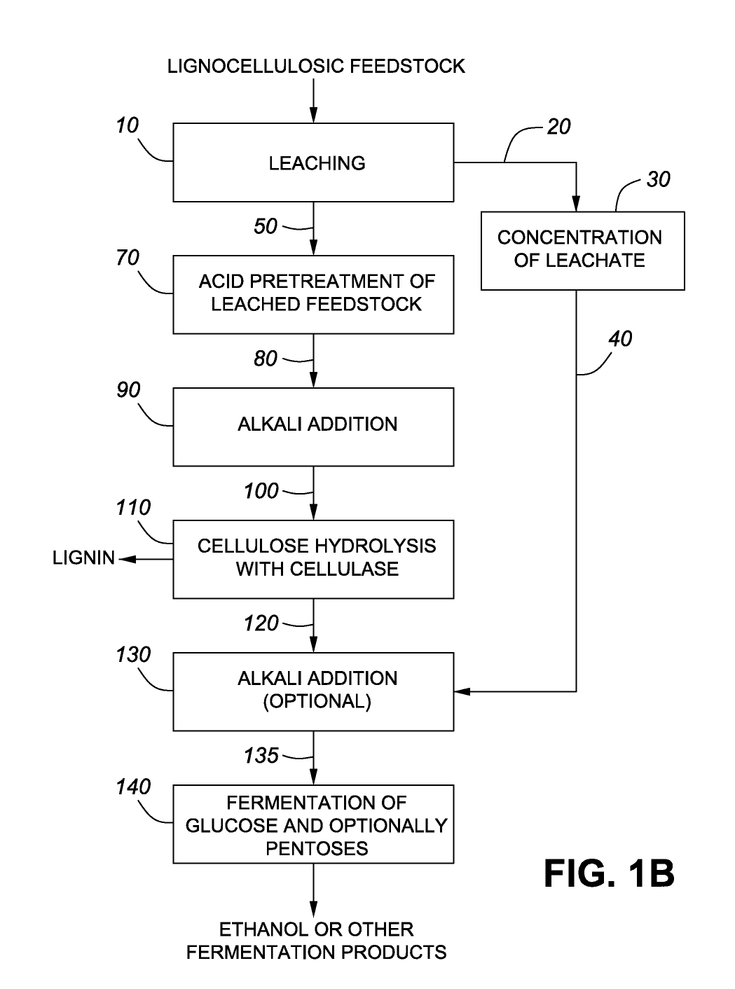

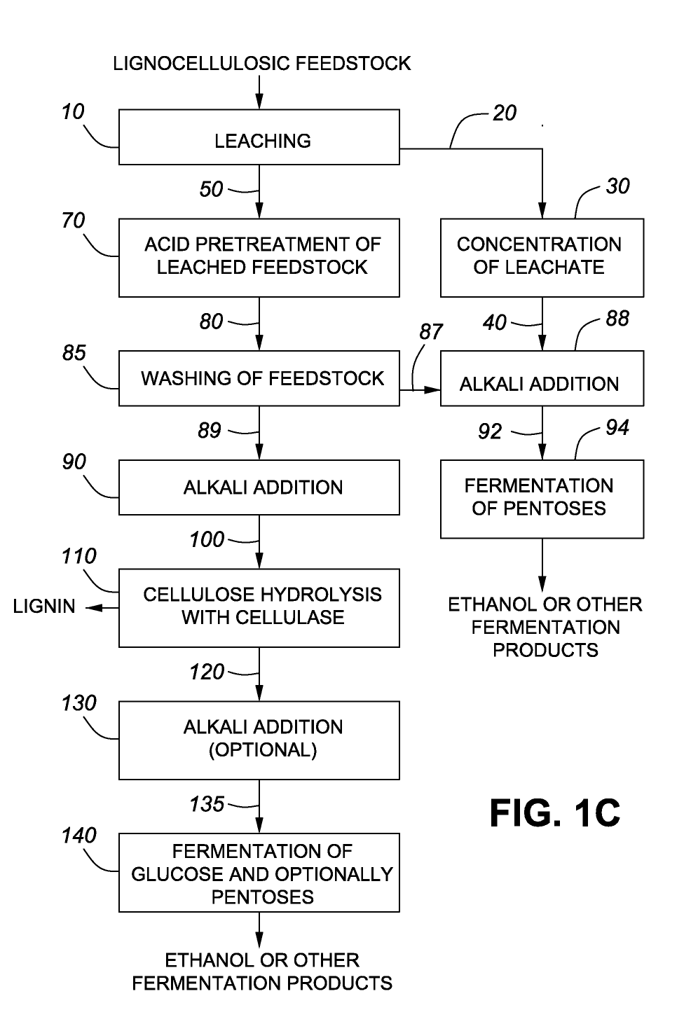

The above-described stages in the lignocellulosic conversion process that may require alkali addition, and where the concentrated leachate may be re-circulated, are summarized in FIGS. 1A, 1B and 1C. It should be understood that these figures are provided for illustrative purposes only and are not intended to be limiting in any manner. That is, the concentrated leachate may be utilized in alkali addition steps required in other known lignocellulosic conversion processes to produce fermentable sugar.

In FIGS. 1A, 1B and 1C, there are shown embodiments of the invention employing leaching, acid pretreatment, cellulose hydrolysis with cellulase enzymes to produce glucose and fermentation of the glucose to produce a fermentation product such as ethanol with the leachate recycle of the invention. Each figure depicts the steps of leaching of the feedstock prior to acid pretreatment, separation of the leachate from the feedstock solids, concentration of the separated leachate and then recirculating the resultant concentrated leachate to a stage of the process requiring alkali addition so as to reduce alkali demand. Like references numbers among the figures depict similar or identical stages or process streams.

As set forth previously, recycle of concentrated leachate can be after pretreatment and prior to cellulose hydrolysis with cellulase enzymes (FIG. 1A), after cellulose hydrolysis with cellulase enzymes and prior to fermentation (FIG. 1B) or after washing the acid pretreated feedstock to obtain hemicellulose-derived sugars and before fermentation of these sugars (FIG. 1C).

Referring now to FIG. 1A, 71 tonnes/h incoming lignocellulosic feedstock composed of wheat straw is subjected to leaching 10. During this leaching step 10, potassium is leached from the lignocellulosic feedstock with an aqueous solution by soaking in water for 15 minutes at pH 8.5, 50° C. An aqueous leachate stream 20 of 180,000 L/h removed from the leached feedstock is then evaporated 30 at 80° C. to produce a concentrated leachate stream 40 of flow 18,000 L/h, and which is at a pH of 9.7. Concentrated leachate stream 40 is then added to alkali addition step 90, as discussed in more detail below.

Leached feedstock solids (63 tonnes/hr) in stream 50 are then fed to dilute acid pretreatment 70. Dilute acid pretreatment 70 hydrolyzes the hemicellulose component of the feedstock under the conditions set forth in Foody, U.S. Pat. No. 4,461,648. Stream 80 contains acid pretreated feedstock that is then subjected to an alkali addition step 90 to adjust its pH to between about 4.8 and about 5.0. According to the embodiment of FIG. 1A, stream 40 comprising concentrated leachate is used to increase the pH of the acid pretreated feedstock in alkali addition 90. Stream 40 may be used as the sole means to increase the pH of the acid pretreated feedstock, as is the case here, or it may be supplemented with other alkali.

Resultant neutralized stream 100 comprising pH adjusted, pretreated feedstock is then sent to cellulose hydrolysis with 24 mg cellulase enzymes per gram cellulose 110 to produce glucose stream 120 at 94 g/L concentration and lignin is separated and removed therefrom. The glucose stream 120 may then be subjected to an optional alkali addition step 130 to adjust its pH to between about 4.0 and about 6.0, thereby producing a pH adjusted glucose stream 135. Fermentation 140 of the glucose stream 135 after alkali addition 130 produces ethanol or other fermentation products as described previously.

FIG. 1B is identical to FIG. 1A except stream 40 comprising concentrated leachate is used to adjust the pH of the glucose stream 120 in alkali addition step 130 prior to fermentation 140 rather than the stream comprising the acid pretreated feedstock 80 as in FIG. 1A.

Referring now to embodiment shown in FIG. 1C, 71 tonnes/h incoming lignocellulosic feedstock at 12% moisture is subjected to leaching 10 to remove potassium salts, as described previously in connection with FIG. 1A. An aqueous leachate stream 20 at a flow of 180,000 L/hr comprising potassium removed from the leached feedstock is then concentrated 30 by evaporation at 80° C. to produce a concentrated leachate stream 40 having a flow rate of 18,000 L/hr and a pH of 9.7. Stream 40 is then added to alkali addition step 88, as discussed in more detail below.

Leached feedstock solids in stream 50 are then fed to acid pretreatment 70 that hydrolyzes the hemicellulose component of the feedstock.

Stream 80 contains acid pretreated feedstock that is then washed 85 to remove hemicellulose sugar released during the pretreatment 70, as well as the acid, thereby producing a wash stream 87 comprising sugar and acid. The wash stream 87 is subjected to alkali addition step 88 to adjust its pH between about 4.5 and about 6.0 prior to fermentation 94 of the hemicellulose sugars. Stream 40 containing concentrated leachate is added to alkali addition step 88 to increase the pH of the wash stream 87 so that it falls within the above-mentioned range. Stream 40 may be used as the sole stream to increase the pH of the wash stream 87 or may be supplemented with other alkali. The neutralized wash stream 92 thus produced may then be fed to the fermentation 94 that converts pentose sugars to ethanol, xylitol or other fermentation products.

The washed feedstock solids in stream 89 from washing step 85 are subjected to an alkali addition 90 to adjust the feedstock pH to between about 4 and about 6. Resultant pH adjusted stream 100 comprising pH adjusted, pretreated feedstock is then sent to cellulose hydrolysis with cellulase enzymes 110 to produce glucose stream 120 and lignin is separated and removed therefrom. The glucose stream 120 may then be subjected to an optional alkali addition step 130 to adjust its pH to between about 4.5 and about 6.0 to produce a stream comprising a pH adjusted glucose stream 135. Fermentation 140 of the glucose in this stream produces ethanol or other fermentation products as described previously.

### Example 2

### Experimental Results for Leaching and Recycle of Leachate to a Pretreated Lignocellulosic Feedstock

Two hundred grams of hammer-milled ½ inch wheat straw with a moisture content of 19.1% was soaked in 1600 g of deionized water at 50° C. for thirty minutes. The straw leachate was then filtered through a glass fiber filter and collected. The resulting leachate contained 0.43% dissolved solids and measured a pH of 8.6. The compositional analysis confirmed the presence of trace levels of xylose and arabinose at about 0.002-0.009 g/L as well as 0.2 g/L sulfate, 0.8 g/L potassium and 0.02 g/L magnesium. The leachate was then boiled and evaporated ten-fold. The resulting pH of the evaporated leachate was 6.57.

Ten milliliters of pretreated wheat straw slurry containing 7.3% solids at a pH of 1.3 was stirred at room temperature. The slurry was obtained from the dilute sulfuric acid pretreatment process detailed above (Foody, U.S. Pat. No. 4,461,648). The pH of the pretreated straw slurry was adjusted using the evaporated leachate prepared as described above. The results obtained are shown in Table 1.

TABLE 1
pH of pretreated wheat straw after addition of evaporated leachate

| mL evaporated | |
|---|---|
| leachate/mL of slurry | pH |
| 0 | 1.34 |
| 0.1 | 1.38 |
| 0.2 | 1.46 |
| 0.3 | 1.56 |
| 0.4 | 1.70 |
| 0.5 | 1.89 |
| 0.6 | 2.06 |
| 0.7 | 2.26 |
| 0.8 | 2.46 |
| 0.9 | 2.67 |
| 1.0 | 2.86 |
| 1.1 | 3.04 |
| 1.2 | 3.20 |
| 1.3 | 3.32 |
| 1.4 | 3.45 |

The pH of the pretreated wheat straw was also adjusted by the combined use of evaporated leachate and sodium hydroxide. Ten milliliters of the pretreated wheat straw slurry prepared as described above was stirred at room temperature and then the concentrated leachate and sodium hydroxide were added. The concentrated leachate was first added to achieve a pH of 2.81 and subsequently sodium hydroxide was added to achieve a pH of 4.91. The results are shown in Table 2 below.

TABLE 2
pH of pretreated wheat straw after addition of evaporated leachate and
sodium hydroxide

| | pH |
|---|---|
| mL evaporated | |
| leachate/mL of slurry | |
| 0 | 1.32 |
| 0.5 | 1.85 |
| 1 | 2.81 |
| g NaOH/L of slurry | |
| 0 | 2.81 |
| 8.0 | 3.05 |
| 16 | 3.3 |
| 24 | 3.55 |
| 32 | 3.78 |
| 40 | 4.02 |
| 48 | 4.25 |
| 56 | 4.46 |
| 64 | 4.67 |
| 72 | 4.91 |

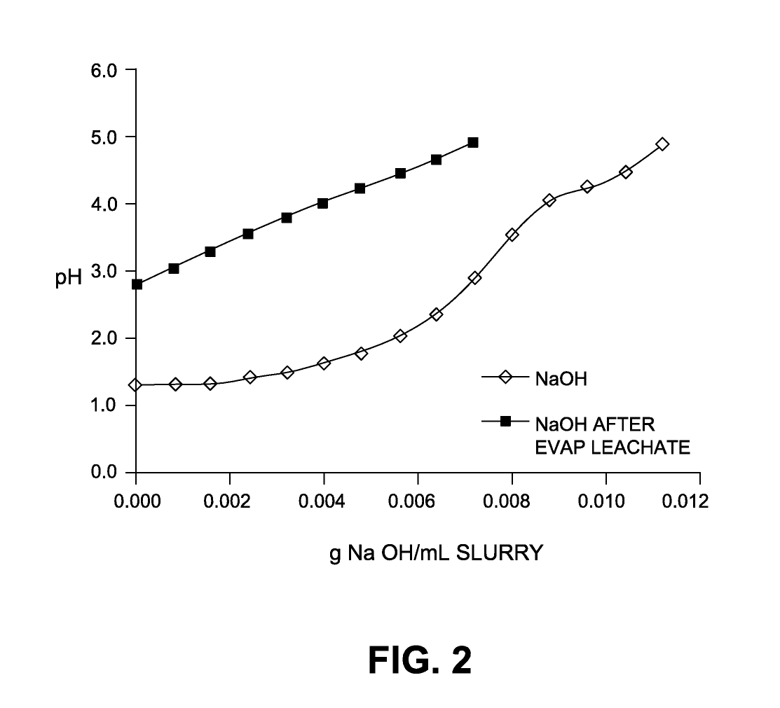

Using the pretreated wheat straw slurry described above, the experiment was repeated using sodium hydroxide only for the pH adjustment. The results are shown in Table 3 and the data from Table 2 and Table 3 is summarized in FIG. 2.

TABLE 3
pH of pretreated wheat straw after addition of sodium hydroxide

| g NaOH/L of slurry | pH |
|---|---|
| 0 | 1.32 |
| 0.8 | 1.33 |
| 1.6 | 1.35 |
| 2.4 | 1.43 |
| 3.2 | 1.53 |
| 4.0 | 1.64 |
| 4.8 | 1.8 |
| 5.6 | 2.03 |
| 6.4 | 2.36 |
| 7.2 | 2.9 |
| 8.0 | 3.55 |
| 8.8 | 4.07 |
| 9.6 | 4.26 |
| 10.4 | 4.49 |
| 11.2 | 4.9 |

The data shows that the use of evaporated leachate, recovered and reused from the process of the invention can be used to replace about 55% or 36% of the sodium hydroxide needed to pH adjust the pretreated wheat straw slurry to pH 4.9.

### Example 4

### Hydrolysis of Pretreated Wheat Straw pH Adjustment Using Evaporated Leachate and Sodium Hydroxide

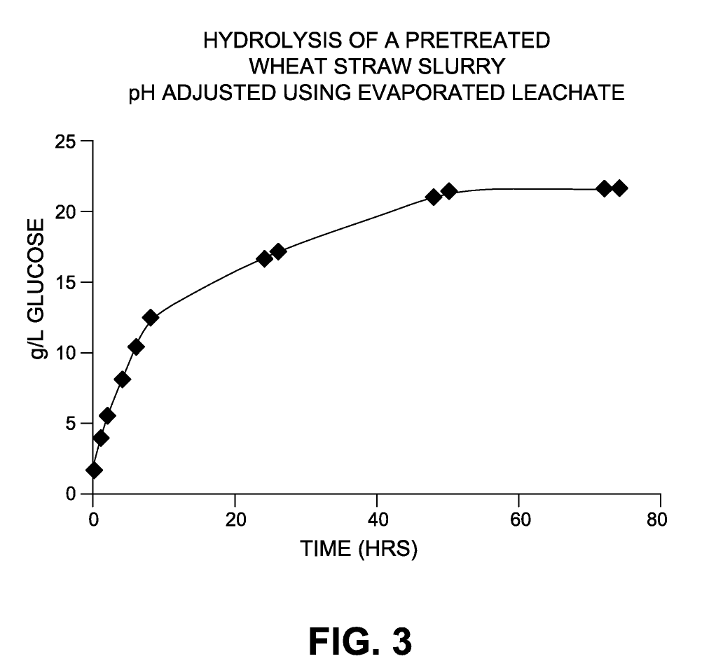

Fifty milliliters of a pretreated wheat straw slurry at an initial pH of 1.3 and containing about 8% undissolved solids, was stirred at room temperature. The pH was adjusted using evaporated leachate until a pH of 2.95 was obtained. This occurred after approximately 50 mL of leachate was added. The evaporated leachate used contained approximately 4.8% dissolved solids including about 7.3 g/L potassium and 0.22 g/L magnesium and trace levels of about 0.01 g/L xylose and glucose. The pH of the slurry was adjusted further to pH 5.02 using 650 μL of 10 M NaOH. The resulting slurry contained about 4% solids. The pH adjusted slurry was transferred to a 250 mL Erlenmyer flask, and pre-incubated at 50° C. for 1 hr, before 30 mg of Iogen cellulase/g cellulose was added. Periodically through the 50° C. incubation, 750 μL samples were withdrawn, heat treated in a 100° C. heat block to deactivate the enzyme, and kept cold until the end of the hydrolysis. As the pH drifted down, the pH was adjusted back to 5 using a 2 M NaOH solution. At the end of the hydrolysis, the samples were clarified by centrifugation and then diluted for the analysis of glucose using a Dionex HPLC and a PA1 column. The results are shown in FIG. 3. The data shows that glucose can be produced from pretreated wheat straw that has been partly pH adjusted to the optimum hydrolysis pH using an evaporated leachate that has been recovered and reused according to the process of the invention.

The above description is not intended to limit the claimed invention in any manner. Furthermore, the discussed combination of features might not be absolutely necessary for the inventive solution.

1. (canceled)

2. A process for producing a fermentation product from a lignocellulosic feedstock comprising the steps of:
(i) leaching the lignocellulosic feedstock with an aqueous solution to remove at least potassium salts from said lignocellulosic feedstock and without significantly hydrolyzing hemicellulose and cellulose, thereby producing a leached feedstock and a leachate;
(ii) removing the leachate from leached feedstock, said leachate comprising at least potassium salt;
(iii) concentrating the leachate comprising the potassium salt to produce concentrated leachate;
(iv) pretreating the leached feedstock with acid to produce an acid pretreated lignocellulosic feedstock;
(v) optionally removing an aqueous stream from the acid pretreated lignocellulosic feedstock comprising the acid and at least xylose, increasing the pH of said aqueous stream to a pH between about 4.0 and 6.0 and then fermenting the xylose to produce a fermentation product;
(vi) adjusting the pH of the acid pretreated lignocellulosic feedstock by alkali addition to produce a pH adjusted feedstock having a pH between about 4 and about 6;
(vii) enzymatically hydrolyzing the pH adjusted feedstock with cellulase enzymes to produce a stream comprising at least glucose;
(viii) optionally increasing the pH of the stream comprising glucose to a pH between about 4.0 and about 6.0 prior to fermenting;
(ix) fermenting the glucose with microorganisms to produce the fermentation product; and
(x) recirculating the concentrated leachate comprising at least potassium salt to
(a) the step of adjusting the pH of the acid pretreated lignocellulosic feedstock;
(b) the step of increasing the pH of the stream comprising glucose;
(c) the aqueous stream comprising xylose prior to fermenting same; or
(d) a combination thereof.

3. (canceled)

4. The process according to claim 2, wherein the concentrated leachate has a pH of between about 7.0 and about 11.0.

5. The process according to claim 2, wherein the pretreating of the leached feedstock with acid comprises hydrolyzing at least a portion of hemicellulose present in said feedstock so as to increase accessibility of cellulose in said feedstock to being hydrolyzed with said cellulase enzymes.

6. The process according to claim 5, wherein the hydrolyzing produces sugar monomers including xylose, glucose, arabinose, mannose, galactose and a combination thereof.

7. The process according to claim 2, wherein the pretreating is conducted at a temperature of between about 160° C. to about 280° C.

8. The process according to claim 2, wherein the pretreating is conducted for between 6 seconds and 3600 seconds.

9. The process according to claim 2, wherein the pretreating is conducted under a pressure of between about 50 psig and 700 psig.

10. The process according to claim 2, wherein the lignocellulosic feedstock comprises feedstock particles and wherein at least about 90% by weight of the particles have a length less than between about 1/16 and about 6 in.

11. The process according to claim 10, wherein the feedstock particles are produced by size reduction of the lignocellulosic feedstock.

12. The process according to claim 2, wherein the re-circulated leachate is supplemented with additional alkali.

13. The process according to claim 2, wherein the concentrating comprises subjecting all of the leachate, or a portion thereof, to one or more concentration steps comprising evaporation, which evaporation is conducted at a temperature of between about 100° C. and about 120° C.

14. The process according to claim 2, wherein the step of enzymatically hydrolyzing further comprises the addition of β-glucosidase.

15. A process for producing glucose from a lignocellulosic feedstock comprising the steps of:
(i) leaching the lignocellulosic feedstock with an aqueous solution to remove at least potassium salts from said lignocellulosic feedstock and without significantly hydrolyzing hemicellulose and cellulose, thereby producing a leached feedstock and leachate;
(ii) removing the leachate from leached feedstock, said leachate comprising at least potassium salt;
(iii) concentrating the leachate comprising the potassium salt to produce concentrated leachate;
(iv) pretreating the leached feedstock to produce a pretreated lignocellulosic feedstock having increased accessibility to hydrolysis of its cellulose component;
(v) adjusting the pH of the pretreated lignocellulosic feedstock to produce a pH adjusted feedstock having a pH between about 4 and about 6;
(vi) hydrolyzing cellulose in the pretreated lignocellulosic feedstock to produce glucose; and
(vii) recirculating the concentrated leachate comprising at least potassium salt to
one or more stages of the process involving alkali addition to adjust the pH of a process stream.

16. A process for producing a fermentation product from a lignocellulosic feedstock comprising the steps of:
(i) leaching the lignocellulosic feedstock with an aqueous solution to remove at least potassium salts from said lignocellulosic feedstock and without significantly hydrolyzing hemicellulose and cellulose, thereby producing a leached feedstock and leachate;
(ii) removing the leachate from leached feedstock, said leachate comprising at least potassium salt;
(iii) concentrating the leachate comprising the potassium salt to produce concentrated leachate;
(iv) hydrolyzing the leached feedstock to produce fermentable sugar in one or more stages;
(v) fermenting the sugar from step (iv) to produce a fermentation broth comprising the fermentation product; and
(vi) recirculating the concentrated leachate comprising at least potassium salt to one or more stages of the process involving alkali addition to adjust the pH of a process stream.

17. The process according to claim 16, wherein the process stream that is subjected to alkali addition is a yeast slurry stream separated from the fermentation broth and wherein the yeast slurry stream is then added back to the fermentation where the fermentable sugar from step (iv) is fermented.
